(19) United States
(12) Patent Application Publication
(10) Pub. No.: US 2004/0009170 A1
(43) Pub. Date: Jan. 15, 2004

(54) Alpha-melanocyte stimulating hormone peptides protection in organ transplantation

(76) Inventors: Stafano Gatti, Como (IT); James M. Lipton, Woodland Hills, CA (US); Anna Catania, Milan (IT)

Correspondence Address: PERKINS COIE LLP, POST OFFICE BOX 1208, SEATTLE, WA, 98111-1208, US

(21) Appl. No.: 10/193,470

(22) Filed: Jul. 10, 2002

(51) Int. Cl. A61K 39/395

(52) U.S. Cl. 424/145.1

(57) ABSTRACT

A composition and method for controlling host response to organ and/or tissue transplantation and grafting. Alpha-Melanocyte Stimulating Hormone protects organ and tissue transplantation by controlling factors within the donor, host and of the organ or tissue to be transplanted. Treatment with α-MSH and/or its derivatives can affect warm and cold ischemia times and thus promotes organ viability. Treatment of the donor, host and of the organ or tissue to be transplanted with an appropriate dosage of α-MSH and/or its derivatives limits biochemical pathways that would normally work to reject an organ and/or tissue transplantation. α-MSH augments successful graft transplantation whether it be allograft or xenograft.

### FIELD OF THE INVENTION

The present invention relates to controlling host response to organ and/or tissue transplantation and grafting.

### BACKGROUND OF THE INVENTION

A list of definitions of certain terms used in this disclosure is included at the end of this section.

With the advance of science has come an increase in the successful treatment of many maladies heretofore believed to be untreatable either because the procedures were such that their practice was impractical and/or because intrinsic limitations pushed such procedures beyond the bounds of science. One field that has experienced recent advancement is that of organ transplantation. The development of diagnostics allowing for antigen matching of organs from diverse sources, as well as improvements in immunosuppression, have allowed the transplantation of such necessary internal organs as the pancreas, kidneys, liver and the heart. However, with this increasing advancement has come an increasing demand for organ transplantation. Although transplantation of organs is becoming more commonplace, rejection of the donated organ by the patient remains a serious problem.

Allograft rejection of vascularized organs is characterized by two main responses. The first is an acute phase response (APR). APR it is a highly complex series of cellular and humoral interactions involving both T lymphocytes, mixed populations of cells and effector mechanisms that ultimately bring about graft failure. T cells initiate and regulate graft rejection. A wide array of effector mechanisms, including alloantibody dependent mechanisms. (B lymphocytes), antigen-specific cytotoxic T cells and a variety of non-specific effector cells, including macrophages, natural killer (NK) cells, polymorphonuclear leukocytes (PMN, e.g., neutrophils, eosinophils) and lymphokine-activated killer (LAK) cells participate in inducing graft destruction.

Hence, a major component of cellular rejection is the result of migration of inflammatory cells into the graft. Lymphocytes, such as those listed above, recognize and react to foreign antigens by undergoing proliferative expansion and initiating humoral events such as antibody formation, cytokine release and the production of pro-inflammatory mediators that in turn recruit and activate other non-specific mediators of the monocyte/macrophage lineage to infiltrate and destroy graft tissue.

The second main response for allograft rejection is a chronic phase response, which is due to the fact that most grafts are subject to ischemic injury due to warm and/or cold ischemia times that result in vasoconstriction of donor arteries followed by reperfusion and the infiltration of the graft by inflammatory cells; primarily, monocytes, macrophages and PMNs. This results in a local, chronic cellular immune response of the endothelium composed of T cells and macrophages that continue to amplify and perpetuate the immune/inflammatory response, further resulting in loss of intact endothelium and function combined with chronic immunologic injury. Both acute and chronic phase response act in relation to the ischemia time involved in pre and intra operative transplantation.

Ischemia time is either warm or cold. Warm ischemia time is the time without the organ or tissue being in an icy environment, an environment commonly used to prolong viability of the organ to be transplanted. The organ or tissue's ability to survive warm ischemia is partially related to the muscle content of the tissue or organ to be transplanted. This is due to muscular tissue's dependence on aerobic glycolysis for energy production. In the absence of an appropriate blood supply, nutrition and clearance, metabolites accumulate, resulting in a sharp decrease in pH. Lactic acid is a well know agent in this process. After two hours of warm ischemia, muscle can fairly readily recover. However, after four hours, the recovery phase is prolonged. After six hours, recovery is unlikely. It is well established that a cold environment slows the accumulation process allowing for longer survival times. But even cold environments lead to deterioration after several hours. There is little that can be done to prevent ischemic injury short of shortening the time a tissue or organ is without an appropriate blood supply. Even the most common measure of placing the tissue in an icy environment is limited to preservation proportional to the cold ischemia time.

Although an icy environment, or cold ischemia time, may prolong viability of the organ to be transplanted, it does so at a risk to tissue. Cold ischemia time is the time that passes while the organ is out of the body and in a cold environment such as that produced by ice, liquid nitrogen, cryogenics, thermal liquid saline solutions, etc. Many of the tissues and/or organs that become available for transplantation require transportation, this increases the time that said tissue is on ice, and consequently lengthens the cold ischemia time. Using current methods, whereby organs are packed in coolers filled with ice and special solutions, organs can only be preserved safely for limited periods of time. The duration depends on the organ. The cold ischemic time for the heart is the shortest. Once it is removed from the donor and has no blood supply, it has a “shelf life” of approximately six hours. Additional tissue damage results upon the reperfusion of the tissue.

The resulting reperfusion of the grafted tissue can result in microtramatic injury to arteriolar intima. This is referred to as transplant ischemia-reperfusion injury. The donor arterial endothelial cells are the primary subjects of transplant ischemia-reperfusion injury. This, as well as or apart from chronic rejection, leads to the gradual deterioration of graft function and is a major threat to long-term survival of transplanted organs, specifically after prolonged cold ischemia times.

Minimization of cold ischemia-reperfusion (CIR) injury is one of the current challenges in organ transplantation for preventing primary organ failure and secondary chronic rejection. The loss of structural intimal integrity, catalytic capacity for respiration, and cytochrome c release constitute early events in CIR. This is accompanied by a reduction of mitochondrial membrane potential. These events are closely associated with the release of various cytokines, chemokines and the expression of adhesion molecules (ICAM-1, VCAM-1 and ELAM-1) and stress proteins (HSP60 and HSP70) that affect ultrastructural changes after cold storage, and which simulate reperfusion. Damaged mitochondria leads to heart injury by the diminished cellular energy status, oxidative stress, disturbance of ion balance, cytochrome c release, initiation of both a cellular and a humoral immune response and eventually the induction of apoptosis.

Hence, save for the two unique situations where organ donation is between identical twins and the special instance of transplantation in individuals with severe combined immunodeficiency disease, all transplant recipients currently require an immunosuppressive regimen to prevent rejection. These immunosuppressive drugs are administered post-transplantation in an attempt to prevent rejection; however, they are notoriously powerful drugs that suppress the body's defenses against infection. Thus, transplantation requires a continued effort to induce acceptance of the graft without paralyzing the body's immune system.

There are various therapies currently used to modulate this response, most using one or more of the following agents: corticosteroids, such as prednisone; cytotoxic drugs, such as azathioprine and cyclophosphamide; x-ray irradiation therapy; anti-lymphocyte and anti-thymocyte globulins; cyclosporine; and monoclonal antibodies such as OKT3, which reacts specifically with the CD3 antigen-recognition structure of human T cells and blocks the T cell effector function involved in allograft rejection. Unfortunately, the side effects of these treatments can equal or surpass the desired effect or indication for use.

For example, corticosteroids may cause decreased resistance to infection, painful arthritis, osteoporosis, and cataracts. Cytotoxic agents may cause anemia and thrombocytopenia, and sometimes hepatitis. The antilymphocytic globulins may cause fever, hypotension, diarrhea, or sterile meningitis. Cyclosporine may cause decreased renal function, hypertension, tremor, anorexia, and elevated low-density lipoprotein levels. OKT3 may cause chills and fever, nausea, vomiting, diarrhea, rash, headache, photophobia, and occasional episodes of life-threatening acute pulmonary edema. It is well known that transplantation is an area of medicine replete with challenges to the host and transplantation specialist.

There is an ever-pressing need for organ donors. In 1996 there were over 2,340 heart transplants, 805 lung transplants, 39 heart and lung transplants and 4,000 liver transplants performed. In 1997 there were over 3,900 heart transplants, 2,700 lung transplants, 235 heart and lung transplants, and 9,600 liver transplants, and the numbers have been steadily growing ever since. More than 78,000 patients are on the national transplant waiting list, including nearly 50,000 who await donor kidneys. While those awaiting heart transplants number about 7,000, only 5,200 heart transplants are performed each year and close to 800 die annually waiting for a heart. Overall, almost 6,000 patients in the U.S. will die due to the limited supply of organs.

A wide source of donor organs are potentially available to various patients in need of a transplant. However, often times the geographic separation between the possible donor and recipient leads to an increased cold-ischemia time (i.e., the time the kidney, for instance, is out of the body on ice being transported about the country), which negatively impacts the viability of the allograft. Further due to positive crossmatch that is typically observed between a highly sensitized organ-recipient and organ-donor, which leads to allograft rejection via both an acute and chronic immune response, only a very small percentage of available donor-organs are actually capable of being transplanted for any given potential organ-recipient. Thus, methods useful for increasing the viability of donor-organs after prolonged cold ischemia times and/or decreasing the adverse affects of antigen cross matching in organ-recipient candidates are drastically needed.

As mentioned, different organs present different warm and cold ischemic times and therefore differing host viability variables. For example, the transplantation of a heart versus a kidney presents different pre, intra and post-operative obstacles. The heart transplant candidate risks dying on the waiting list while waiting for a prospectively matched compatible organ. A patient in chronic renal failure may survive for years on a dialysis machine while awaiting a kidney transplant. Where the heart patient would most likely perish due to a rejection of the transplanted heart, if the transplanted kidney were rejected, the patient can be returned to dialysis treatment. In addition, a heart transplant donor is typically identified only hours before the actual transplantation is to take place, and there is insufficient time to perform the crossmatch assays that are generally employed to screen for graft/host histocompatibility. Heart transplant candidates are thus at risk of undergoing a hyperacute rejection from an incompatible organ crossmatched retrospectively.

Because no alternate course of treatment is available, physicians are highly selective in choosing potential heart transplant donors. In view of the heightened scrutiny involved in transplanting a heart, the inventive method of increasing the likelihood of a negative crossmatch dramatically increases the pool of heart organs that the patient will not reject and therefore provides the patient with an increased likelihood of survival.

The present invention is particularly suitable for the transplantation of hearts, which have a reduced cold-ischemia time tolerability. In this regard, an allograft or xenograft is removed from a donor subject. Once the allograft or xenograft is removed it may then be placed in a cold environment so as to prevent warm ischemia. Ice, liquid nitrogen, cryogenics, cold saline, or any other such method of producing a suitably cold environment may produce this cold environment. To date, no significant change in tolerability to either cold or warm ischemia times have been achieved with respect to allograft or xenograft organs.

Thus, current technology provides only a small window of opportunity to transport and transplant an organ, thereby greatly limiting the availability of organs to those in need. The ability for a tissue or organ to survive a longer preservation time and/or survive transplantation despite antigen-cross match, would allow the sharing of a greater number of organs across greater distances, thereby allowing more patients to benefit from life-saving transplants and providing transplant teams with greater time to perform a complete range of tests on organs that are currently not considered suitable for transplantation, such as those from older donors or those with questionable function. For that matter, organs with “marginal” suitability for transplantation may be considered, thereby decreasing the necessity of long transportation of strictly compatible organs.

With the increasing need for organ transplantation and the use of “marginal” organs, novel approaches are sought to increase the efficiency and survival of transplanted tissues. According to the presently described methods, the idea that treatment with the anti-inflammatory peptide α-melanocyte stimulating hormone (α-MSH) may protect allografts and prolong their survival was tested.

Definitions:

The following terms common to this specification and claims are defined here. Terms used less commonly in this specification will be defined as they are used.

α-Melanocyte stimulating hormone (α-MSH): An ancient, endogenous 13 amino acid peptide produced by post-translational processing of proopiomelanocortin (POMC).

α-MSH(1-13)(Sequence ID No. 1): Refers to an amino acid sequence containing SYSMEHFRWGKPV.

α-MSH(11-13)(Sequence ID No. 3): Refers to an amino acid sequence containing KPV.

α-MSH(8-13)(Sequence ID No. 4): Refers to an amino acid sequence containing RWGKPV.

α-MSH derivative: refers to any portion of α-MSH or substitutions of amino acids within α-MSH with biologically functionally equivalent amino acids with resulting differences in efficacy and potency. Nle⁴DPhe⁷-α-MSH (“NDP-α-MSH”)(Sequence ID No. 2 which contains amino acid sequence SYS(Nle)EHFRWGKPV) is an example in which the resulting peptide is markedly more potent than the natural molecule.

Allograft: An organ or tissue transplanted from one individual to another of the same species; e.g., human to human.

Allograft failure: Failure of an allograft transplantation.

Before Transplantation: Refers to the use of the claimed method prior to removal of the organ to be transplanted, use of the claimed method during transportation of the organ once removed from the donor and/or use of the claimed method with respect to the host prior to transplantation of the organ in the host.

Chronic allograft failure: The gradual failure of a transplanted organ.

Cold Ischemia Time: The time interval beginning when an organ is cooled with a cold perfusion solution at the organ procurement surgery and ending when the organ is re-perfused at implantation.

Crossmatch: A test preformed to detect antibodies in a potential recipient's blood against antigens on the surface of a potential donor's cells. A positive crossmatch means that the recipient has antibodies against the donor's cells. With few exceptions, a positive crossmatch makes successful transplantation between that donor and recipient impossible.

End-Stage [organ] Failure or Chronic [organ] Failure or End-Stage [organ] Disease: The irreversible and permanent pathological condition of an organ or organ system commonly resulting in organ replacement. Typically, one may see a particular condition referred to as a simple letter designation, i.e. ESRD for End-Stage Renal Disease. For kidney (renal), liver (hepato), heart (cardio), lung (pulmano), pancreas and intestine failure, there may be an option of transplantation.

Graft: The transplantation of a tissue. Autograft refers to that tissue transplanted from one area of an individual to a different area of the same individual. Allograft refers to tissues or organs transplanted from another of the same species; e.g., human to human. In this specification, graft is used interchangeably with allograft.

Human Leukocyte Antigen System (HLA System): Human Leukocyte Antigens (HLA), also known as histocompatibility antigens, are molecules found on all nucleated cells in the body. Histocompatibility antigens help the immune system recognize whether or not a cell is foreign to the body. These antigens are hereditary. Human leukocyte antigens are used to determine the compatibility of organs for transplantation from one individual to another. The major groups of HLA antigens are HLA-A, HLA-B, and HLA-DR.

Immunosuppression: The suppression of the immune response, usually with medications, to prevent the rejection of a transplanted organ or tissue. Medications commonly used to suppress the immune system after transplantation include prednisone; prednisolone, methylprednisolone, azathioprine, mycophenalate mofetil, cyclosporine, tacrolimus, sirolimus, and antibodies developed to interfere with the function of the immune system itself.

Induction immunosuppression: The use of intensified immunosuppression immediately after transplantation.

Tissue Type: An individual's unique combination of HLA antigens is called their tissue type. Matching for tissue type is critical to transplantation. Each waitlisted patient's tissue type is entered into a central computer maintained by the Organ Procurement and Transplantation Network (“OPTN”)

Warm ischemia time: The time without the organ or tissue to be transplanted being in an icy environment, an environment commonly used to prolong viability of the organ to be transplanted.

Xenograft: An organ, tissue, cell, or body fluid transplanted, implanted, or infused from a member of another species.

Xenotransplant product(s): Live cells, tissues, or organs used in xenotransplantation.

Xenotransplantation: Any procedure that involves the transplantation, implantation, or infusion into a recipient of one species of live cells, tissues, or organs from a different species. In the case of humans, a human receives tissues or live cells from a different animal source. Xenotransplantation also refers to host body fluids, cells, tissues, or organs that have had ex vivo contact with live, non-host animal cells, tissues, or organs.

As used herein, “transplant” or various grammatical forms thereof, means the physical act of providing a patient with tissues from a source distinct from the patient. The transplant can be either a primary graft or a regraft. Methods for conducting the transplantation procedures for a variety of body organs are well known in the art. See, for example, Danovitch, G., Handbook of Kidney Transplantation, Little Brown & Co., Boston, Mass. 1992.

A more in-depth understanding of the concept of “crossmatching” refers to assays that determine the presence of anti-HLA antibodies in a candidate transplant patient that are reactive with the HLA antigen on the cells of another individual (i.e., a potential organ-donor). A “positive” crossmatch, or reference to a “histoincompatible” organ, refers to the presence of anti-HLA antibodies that are immunoreactive with the HLA-antigen on the cells of the potential organ-donor, such that transplantation of an allograft from a donor with a positive crossmatch will frequently result in a hyperacute, acute, or chronic rejection of the allograft, but usually the former.

In contrast, “negative” crossmatch refers to the absence of anti-HLA antibodies that are immunoreactive with the HLA-antigen on the cells of the potential organ-donor, such that upon transplant of an allograft from a donor, the allograft is not likely to be rejected. Higher than normal levels of anti-HLA antibodies in a potential transplant patient can be determined by a variety of methods well-known in the art. A patient displaying greater than about 50% is said to be “highly” sensitized. PRA refers to the percentage of individuals in an HLA typed panel (i.e., potential organ-donors) with which blood serum from a given patient will immunoreact. For example, a patient's serum that reacts with (i.e., is cytotoxic to) positive lymphocytes from 95 of 100 individuals is said to have a PRA value of 95%.

### SUMMARY OF THE INVENTION

α-MSH is an ancient endogenous polypeptide that, while it may not directly affect immunosuppression, does reduce reperfusion injury. The present invention relates to a composition of α-MSH, specifically Nle⁴Dphe⁷-α-MSH and an immunosuppressive agent, and methods using the same for reducing graft rejection. In a particular aspect, the invention relates to methods to augment or serve as an immunosuppressive in a potential transplant host so that host may be more amenable to transplant with donor organs obtained from a variety of donors, including histoincompatible donors and/or donors of different species. Specifically, the invention relates to the prevention of reperfusion injury and both acute and chronic rejection via the inhibition of NF-κB by α-MSH. Thus, this invention is useful in treating and/or preventing pathological conditions associated with unwanted NF-kB activation. The inhibition of NF-kB activation, inter alia, by the disclosed compositions and methods of the present invention are useful for prolonging the survival of allografts. The administration of α-MSH based compositions leads to a decrease in leukocyte infiltration, primarily with respect to neutrophils, monocytes, activated and memory T cells, B cells, NK cells and eosinophils. Furthermore, the above administration should also result in a decrease in the activation of inflammatory elements such as endothelin (specifically Endothelin I), nitric oxide synthase-II, monocyte chemoattractant protein 1 (MCP-1), interferon-γ, TNFα (tumor necrosis factor-α), transforming growth factor-β, RANTES, ICAM-1, VCAM-1, FasL, IL-10, IL-8, fMLP, PDGF-B, as well as other inflammatory cytokines, chemokines and adhesion molecules.

A composition consisting of an α-MSH and/or an α-MSH derivative and an immunosuppressive agent in a biologically acceptable carrier may then be administered to a host subject followed by actual transplantation. The α-MSH composition may be administered before, after and/or during the actual transplantation procedure. Preferably, and in accordance with the present invention, we have discovered that transplant candidates can be treated prior to transplantation so as to improve the likelihood of successful transplantation. Due to the high patient tolerability and lack of side effects, pre, intra and post transplant treatment with α-MSH and/or its derivatives spare the transplant host the well know perils associated with current chemotherapeutic agents. In combination with those chemotherapeutic agents, less of a particular agent is required and less duration of immunosuppression.

In another aspect of the invention the α-MSH peptides are administered to the organ donor prior to removal of the organ to be transplanted. Further, and in another aspect of the invention, an organ, once removed, can be transported while being treated with the α MSH peptides using topical and or invasive administration. The claimed method is also intended for use in the host prior to transplantation of the donated organ.

The improvement is further accomplished by increasing the likelihood of a negative crossmatch between the transplant host and the organ-donor. The methods for transplanting an allograft in a patient are well known in the art. The administration may be by any suitable route including but not limited to parenteral, oral, anal, mucous membrane transfer and trans-dermal patch. A preferable administration route is via intraperitoneal injection.

The procedure may further be enhanced by the addition of immunosuppressive treatments administered before, after or in conjunction with the administration of the described composition. Hence, the invention methods are useful to expand the available source of donor organs which are acceptable for a given transplant recipient. The invention methods permit an immunosuppressed patient to be successfully immunosuppressed and subsequently transplanted with a crossmatch negative, but histoincompatible, donor-organ. Overall, the present invention improves the prognosis of a transplant recipient for long-term survival (“actuarial graft survival”), and reduces the need for immunosuppressive treatment. Moreover, the present invention prevents infection and does not add to the host's immunosuppressive load, e.g., does not increase the risk of infection due to immunosuppression. In addition, the invention compositions and methods reduce the time that potential transplant candidates spend waiting for a compatible, crossmatch negative donor. Further, the composition and methods of the present invention are also useful for the prevention of irregular cellular apoptosis.

Preferably, subjects contemplated for application of the invention composition and methods are mammals including humans, domesticated animals, and primates. Typically, subjects in need of a transplantation procedure are those who have a higher than normal level of anti-HLA antibodies that are reactive against foreign tissue. Many of these subjects, will typically have been exposed to blood products (i.e., dialysis patients), or will have experienced pregnancy.

### BRIEF DESCRIPTION OF THE DRAWINGS

FIG. 1. Kaplan-Mayer survival curves for transplanted hearts in untreated- and NDP-α-MSH-treated rats. Median survival was 6 and 10 days, respectively.

p<0.001

FIG. 2. Histopathological score of heart grafts harvested 1 and 4 days after transplantation.

**p<0.01; *** p<0.001

FIG. 3. Histopathology of heart grafts harvested 1 (top) and 4 (bottom) days after transplantation. Graft infiltrating cells are immunostained with anti-ED 1 antibody. A and C untreated; B and D NDP-α-MSH-treated.

FIG. 4. Treatment associated changes in gene expression in heart grafts harvested 1 day after transplantation.

* p<0.05; ** p<0.01; *** p<0.001

FIG. 5. Treatment associated changes in gene expression in heart grafts harvested 4 days after transplantation.

* p<0.05; ** p<0.01; *** p<0.001

FIG. 6. Changes over time in plasma concentrations of the NO metabolite NO₂ after heterotopic heart transplantation in untreated- and NDP-α-MSH-treated rats.

* p<0.05; *** p<0.001

### DETAILED DESCRIPTION OF THE PREFERRED EMBODIMENTS

α-Melanocyte stimulating hormone (α-MSH) is an ancient, endogenous 13-amino acid peptide produced by post-translational processing of proopiomelanocortin (POMC). Its amino acid sequence is identical in mammals and highly conserved across animal species, extending into invertebrates. Eberle A N, “The Melanotropins,” Basel, ed. S. Karger (1988). The peptide is produced by the pituitary and by many extrapituitary cells, including monocytes, astrocytes, gastrointestinal cells, and keratinocytes. Catania A, Lipton J M, “α-Melanocyte stimulating hormone in the modulation of host reactions,” Endocr. Rev., 14:564-576 (1993). α-MSH also modulates host reactions. Catania A, Lipton J M, “α-Melanocyte stimulating hormone in the modulation of host reactions,” Endocr. Rev., 14:564-576 (1993); Catania A, Airaghi L, Colombo G, Lipton J M, “α-MSH in normal human physiology and disease states,” Trends Endocrinol. Metab., 11:304-308 (2000). Additionally, the peptide has been shown to have potent antipyretic properties. Murphy M T, Richards D B, Lipton J M, “Antipyretic potency of centrally administered α-melanocyte stimulating hormone,” Science, 221:192-193 (1983). Anti-inflammatory effects of α-MSH have been well documented. Hiltz M E, Lipton J M, “Anti-inflammatory activity of a COOH-terminal fragment of the neuropeptide α-MSH,” FASEB J., 3:2282-2284 (1989). The peptide also functions as an immunomodulatory agent. Grabbe S, Bhardwaj R S, Malinke K, Simon M M, Schwarz T, Luger T A, “α-Melanocyte-stimulating hormone induces hapten-specific tolerance in mice,” J. Immunol., 156:473-478 (1996). It has likewise been established that the peptide has antifungal and antimicrobial effects. Cutuli M G, Cristiani S, Lipton J M, Catania A, “Antimicrobial effects of α-MSH peptides,” J. Leukoc. Biol., 67:233-239 (2000). Given in pharmacological concentrations, α-MSH is extremely effective in preclinical treatment of local and systemic inflammatory disorders including sepsis syndrome, acute respiratory distress syndrome, rheumatoid arthritis, inflammatory bowel disease, and encephalitis. Lipton J M, Catania A, “Anti-inflammatory actions of the neuro-immunomodulator α-MSH,” Immunol. Today, 18:140-145 (1997). Studies on the ischemic brain have yielded similar findings. Huh S K, Lipton J M, Batjer H H, “The protective effects of α-melanocyte stimulating hormone on canine brainstem ischemia,” Neurosurgery, 40:132-139 (1997).

α-MSH and/or its derivatives may be used before transplantation in the host, donor or directly on or in the organ to be transplanted once the organ has been removed from the donor. Methods of administration, with respect to the donor or host include, but are not limited to, oral, anal, parenteral, intravascular, intrarterial, topical, transdermal, vaginal, intratracheobronchial mucosal intraperitoneal and intracerbroventricular. These same methods of administration are contemplated for this invention during transplantation as well. With respect to the organ to transplanted, α-MSH and/or its derivatives may be administered topically or through any non-traumatic invasive measure known in the art.

The anti-inflammatory effects of A-MSH are mainly exerted through increases in cell cAMP and inhibition of NF-κB-dependent gene transcription as described in the following references: Manna S K, Aggarwal B B, “α-Melanocyte-stimulating hormone inhibits the nuclear transcription factor NF-κB activation induced by various inflammatory agents,” J. Immunol., 161:2873-2880 (1998); Ichiyama T, Zhao H, Catania A, Furukawa S, Lipton J M, “α-Melanocyte-stimulating hormone inhibits NF-κB activation and I κBα degradation in human glioma cells and in experimental brain inflammation,” Exp. Neurol., 157:359-365 (1999). In unstimulated cells, this essential transcription factor is sequestered within the cytoplasm and binds to the inhibitory molecule IκB. Barnes P J, Karin M, “Nuclear factor-KB: a pivotal transcription factor in chronic inflammatory diseases,” New Engl. J. Med., 336:1066-1071 (1997). Upon cell stimulation, IκB is phosphorylated and rapidly degraded. NF-κB is then released from IκB and translocated to the nucleus where it induces gene expression by binding to various DNA recognition sites. Barnes P J, Karin M, “Nuclear factor-KB: a pivotal transcription factor in chronic inflammatory diseases,” New Engl. J Med., 336:1066-1071 (1997). Previous research indicates that α-MSH prevents IκB degradation and, consequently, reduces translocation of NF-κB to the nucleus. Manna S K, Aggarwal B B, “α-Melanocyte-stimulating hormone inhibits the nuclear transcription factor NF-κB activation induced by various inflammatory agents,” J. Immunol., 161:2873-2880 (1998); Ichiyama T, Zhao H, Catania A, Furukawa S, Lipton J M, “α-Melanocyte-stimulating hormone inhibits NF-κB activation and I κB degradation in human glioma cells and in experimental brain inflammation,” Exp. Neurol., 157:359-365 (1999).

Therefore, α-MSH should be useful for treatment of pathologic conditions in which activation of NF-κB is prominent. One such condition is graft rejection. NF-κB enhances transcription of genes, the products of which are critical for inflammation and immunity. Barnes P J, Karin M, “Nuclear factor-KB: a pivotal transcription factor in chronic inflammatory diseases,” New Engl. J Med., 336:1066-1071 (1997). It appears that NF-κB-dependent molecules contribute to reperfusion injury and acute rejection. Cooper M, Lindholm P, Pieper G, Seibel R, Moore G, Nakanishi A, Dembny K, Komorowski R, Johnson C, Adams M, Roza A, “Myocardial nuclear factor-KB activity and nitric oxide production in rejecting cardiac allografts,” Transplantation, 66:838-844 (1998); Feeley B T, Miniati D N, Park A K, Hoyt E G, Robbins R C, “Nuclear factor-κB transcription factor decoy treatment inhibits graft coronary artery disease after cardiac transplantation in rodents,” Transplantation, 70:1560-1568 (2000). Such molecules include cytokines, immunoreceptors, cell adhesion molecules, acute phase proteins, and inducible nitric oxide synthase (NOS II). Because production of all these molecules is modulated by α-MSH, Lipton J M, Catania A, “Anti-inflammatory actions of the neuro-immunomodulator α-MSH,” Immunol. Today, 18:140-145 (1997); Star R A, Rajora N, Huang J, Stock R C, Catania A, Lipton J M, “Evidence of autocrine modulation of macrophage nitric oxide synthase by α-melanocyte-stimulating hormone,” Proc. Natl. Acad. Sci. USA, 92:8016-8020 (1995); Taherzadeh S, Sharma S, Chhajlani V, Gantz I, Rajora N, Demitri M T, Kelly L, Zhao H, Catania A, Lipton J M, “α-MSH and its receptors in regulation of inflammatory tumor necrosis factor-α (TNF α) by human monocyte/macrophages,” Am. J Physiol., 276:R1289-R1294 (1999). It follows that this peptide may be used in combination with immunosuppressive agents to inhibit allograft rejection.

Additionally, analogs of the α-MSH peptide, and specific to this invention, [Nle⁴, Phe⁷]α-MSH, may greatly enhance the anti-inflammatory potency, duration and efficacy of the peptide without adding to unwanted side-effects. The α-MSH analog [Nle⁴, Phe⁷]α-MSH is resistant to inactivation by proteolytic enzymes and has prolonged activity in vivo and in vitro. It is 10- to 1000-times more active than the natural molecule depending upon the assay employed. Oxidation of Met⁴ in the natural sequence leads to sulphoxide α-MSH which is inactive. Replacement of Met⁴ with the isosteric norleucine (Nle⁴) greatly reduces such an inactivation pathway. Further, substitution of the Phe⁷ with its D isomer increases anti-inflammatory potency, duration and efficacy of the molecule by a factor of approximately 10.

According to the procedure described in detail below, α-MSH was administered during experimental heart transplantation in rats. Donor cardiac grafts (Brown Norway) were transplanted into the abdomen of recipient (Lewis) rats. Treatments consisted of intraperitoneal injections of Nle⁴DPhe⁷-α-MSH (NDP-α-MSH) or saline from the time of transplantation until sacrifice or spontaneous rejection.

Allografts were removed on day 1, 4, or upon rejection, and examined for histopathology and expression of molecules prominent in reperfusion injury, transplant rejection, and apoptosis. α-MSH treatment caused a significant increase in allograft survival and a marked decrease in leukocyte infiltration. Further, expression of molecules such as endothelin 1, chemokines, and adhesion molecules, which are involved in allograft rejection, were significantly inhibited in α-MSH-treated rats. The results show that the protection of the allograft from early injury by α-MSH can postpone and ultimately eliminate host rejection. Addition of this early protection with the peptide to usual treatment with immunosuppressive agents improves the success of organ transplants.

Therefore, the primary aim of research leading to the development of the present invention was to determine whether α-MSH treatment protects the allograft and prolongs survival in experimental heart transplantation, in the absence of immunosuppressive therapies. The data showed that α-MSH did prolong allograft survival. To determine the mechanism underlying this beneficial effect, we compared changes over time in histopathology and gene expression in heart grafts from treated and untreated animals. Gene transcripts were selected in order to have a broad overview of reperfusion injury, transplant rejection and apoptosis pathways that were potentially altered by the peptide. Selected gene transcripts included: endothelin 1 (ET-1); nitric oxide synthase-II (NOS II); monocyte chemoattractant protein 1 (MCP-1); regulated upon activation normal T-cell expressed and secreted (RANTES); intercellular adhesion molecule-1 (ICAM-1); vascular adhesion molecule-1 (VCAM-1); Fas ligand (FasL); interferon-γ (IFN-γ); tumor necrosis factor-α (TNF-α); interieukin-1 β (IL-1β); platelet derived growth factor B-chain (PDGF-B); and transforming growth factor-β (TGF-β). Finally, as a measure of systemic inflammation and its modulation by α-MSH, we determined plasma concentrations of the nitric oxide metabolites nitrite/nitrate.

The compositions of the present invention may be formulated and used as tablets, capsules, or elixirs for oral administration; as suppositories for rectal or vaginal administration; sterile solutions and suspensions for parenteral administration; creams, lotions, or gels for topical administration; aerosols for intratracheobronchial administration; and the like. Preparations of such formulations are well known to those skilled in the pharmaceutical arts. The dosage and method of administration can be tailored to achieve optimal efficacy. Pharmaceutical titration to achieve maximum benefit of medicinal compounds is well known in the art.

For administration, the therapeutic composition will generally be mixed prior to administration with a non-toxic, biologically compatible carrier. Usually, this will be an aqueous solution, such as normal saline or phosphate-buffered saline (PBS), Ringer's solution, Ringer's lactate or any isotonic physiologically acceptable solution for administration by the chosen means. Preferably, the solution is sterile and pyrogen-free, and is manufactured and packaged under current Good Manufacturing Processes (GMP's) as approved by the FDA. The clinician of ordinary skill is familiar with appropriate ranges for pH, tonicity, and additives or preservatives when formulating pharmaceutical compositions for administration. In addition to additives for adjusting pH or tonicity, the therapeutic agent may be stabilized against aggregation and polymerization with amino acids and non-ionic detergents, polysorbate, or polyethylene glycol.

In one embodiment of the above invention, the α-MSH composition is administered orally. Each oral composition according to the present invention may additionally comprise inert constituents including biologically compatible carriers, dilutents, fillers, wetting agents, suspending agents, solubilizing or emulsifying agents, salts, flavoring agents, sweeteners, aroma ingredients or combinations thereof, as is well-known in the art. Liquid dosage forms may include a liposome solution containing the liquid dosage form. As known by those skilled in the art, suitable forms for suspending liposomes include emulsions, pastes, granules, compact or instantized powders, suspensions, solutions, syrups, and elixirs containing inert dilutents, such as purified water.

Tablets or capsules may be formulated in accordance with conventional procedures employing biologically compatible solid carriers well known in the art. For example, a pharmaceutical preparation may contain the composition dissolved in the form of a starch capsule, or hard or soft gelatin capsule which is coated with one or several polymer films, in accordance with U.S. Pat. No. 6,204,243 which is fully incorporated as if fully set out herein. Undesired dissolution of the capsule shell in the area of the stomach or upper small intestine is prevented by coating the external capsule wall with a polymer film. The choice and usage of appropriate polymers, including additional materials such as softeners and pore-forming agents, control the site of dissolution of the capsule and the release of solution containing the active agent.

Preparation of the composition may also include dissolving the composition in a solvent, which is suitable for encapsulation into starch or gelatin capsules, or in a mixture of several solvents and, optionally, solubilizers and/or other excipients. The solution is then filled into starch capsules, or hard or soft gelatin capsules in a measured dose, the capsules are sealed, and the capsules are coated with a solution or dispersion of a polymer or polymer mixture and dried. The coating procedure may be repeated once or several times.

The solvents that are appropriate for dissolving the active agent are those that are biologically compatible with the host subject and in which the composition dissolves. Examples of these are ethanol, 1,2-propylene glycol, glycerol, polyethylene glycol 300/400, benzyl alcohol, medium-chained triglycerides and vegetable oils.

Furthermore, medicament excipients may be added to the solution. Examples of such excipients are mono-/di-fatty acid glycerides, sorbitan fatty acid esters, polysorbates, lecithin, sodium lauryl sulphate, sodium dioctylsulphosuccinate, aerosol and water-soluble cellulose derivatives. Mixtures of solvents and excipients may also be used. The soft or hard gelatin capsule may be coated with one or several polymer films, whereby the targeted capsule dissolution and release of the therapeutically effective composition is achieved through the film composition. The polymer or a mixture of polymers is dissolved or dispersed in an organic solvent or in a solvent mixture. For example, solvents include ethanol, isopropanol, n-propanol, acetone, ethyl acetate, methyl ethyl ketone, methanol, methylene chloride, propylene glycol monomethyl ether and water. See, in general, Remingtons's Pharmaceutical Sciences (18^{th} Ed. Mack Publishing Co. 1990).

The properties of the polymer films may be further influenced by additions of pore-forming agents and softeners. Suitable pore-forming agents to form open pores, and thus to increase the diffusion rate through the polymer coating, are water-soluble substances, including lactose, saccharose, sorbitol, mannitol, glycerol, polyethylene glycol, 1,2-propylene glycol, hydroxypropyl cellulose, hydroxypropyl methylcellulose, methylcellulose, as well as mixtures thereof. Softeners include alkyl esters of citric acid, tartaric acid and 1,8-octanedi-carboxylic acid, triethyl citrate, tributyl citrate, acetyl triethyl citrate, dibutyl tartrate, diethyl sebacate, dimethyl phthalate, diethyl phthalate, dioctyl phthalate, castor oil, sesame oil, acetylated fatty acid glycerides, glycerol triacetate, glycerol diacetate, glycerol, 1,2-propylene glycol, polyethylene glycols and polyoxyethylene-polypropylene block copolymers, PEG-400 stearate, sorbitan mono-oleate, and PEG-sorbitan mono-oleate.

When administration is parenteral, such as intravenous on a daily basis, injectable pharmaceuticals may be prepared in conventional forms, as aqueous or non-aqueous solutions or suspensions; as solid forms suitable for solution or suspension in liquid prior to injection; or as emulsions. Examples of non-aqueous solvents are propylene glycol, polyethylene glycol, vegetable oils such as olive oil, and injectable organic esters such as ethyl oleate. Examples of suitable excipients are water, saline, dextrose, mannitol, lactose, lecithin, albumin, sodium glutamate, cysteine hydrochloride, or the like. In addition, the injectable pharmaceutical compositions may contain minor amounts of non-toxic auxiliary substances, such as wetting agents, pH buffering agents, and the like. If desired, absorption-enhancing preparations (e.g., liposomes) may be utilized.

The effective amount of the biologically compatible composition to be given to a particular host subject will depend on a variety of factors, several of which will vary from subject to subject. The composition should be administered in such way that it is present at a sufficient concentration to adequately provide a therapeutic benefit. Dosage of the therapeutic will depend on the type of treatment, route of administration, nature of the therapeutic, sensitivity of the cell to the therapeutic, etc. Factors that vary from patient to patient include the patient's age, condition, sex, extent of the disease, and other variables. Utilizing LD₅₀ animal data, and other information available for the administration of such compositions, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions that are cleared rapidly from the body may be administered at higher doses, or in repeated doses, in order to maintain therapeutic concentrations.

The therapeutic may be administered to the subject in a single administration, or it may be administered in a series of administrations to reduce the toxicity of a chosen composition. A lower concentration of the therapeutic over a long period of time may be most effective, or a higher concentration over a short period of time may be preferred. Using ordinary skill, the competent clinician will be able to optimize the dosage of a particular therapeutic composition in the course of routine clinical trials.

### EXAMPLE 1

### Experimental Cardiac Tissue Transplantation in Rats

Adult inbred Brown Norway (donor) and Lewis (recipient) male rats, weighing 200-300 g were used in this study (Charles River, Calco, Italy). Animals were maintained at the animal care facilities of the Department of Hepatology, Ospedale Maggiore di Milano, Italy, under standard temperature, humidity, and time-regulated light conditions. Water and food were provided ad libitum. All animals received care in compliance with the Principles of Laboratory Animal Care, formulated by the National Society of Medical Research, and the Guide for the Care and Use of Laboratory Animals, prepared by the National Academy of Sciences and published by the National Institutes of Health (NIH Publication No. 8623, revised 1985).

Before transplantation, animals were anesthetized with an intraperitoneal injection of 125-mg/kg ketamine. During anesthesia, heart rate, ventilation rate, and temperature were closely monitored. Donor cardiac grafts (Brown Norway) were transplanted into the abdominal cavity of the recipient (Lewis) rats using the technique described by Ono and Lindsey. Ono K, Lyndsey E S, “Improved technique of heart transplantation in rats,” J. Thorac. Cardiovasc. Surg, 57:225-229 (1968). The heart was maintained at 10-14° C. during the implantation period by wrapping it in cold gauze irrigated with cold saline (4° C.). To minimize variability among experiments, the duration of the surgical implantation was standardized at 40 min for all studies. All cardiac transplants had good initial contractile function. There were no early deaths. Graft function was monitored by palpation through the abdominal wall twice daily. Allograft rejection was defined by loss of palpable contractile activity and was confirmed by direct inspection laparotomy.

Treatments consisted of intraperitoneal injections of 0.5 ml saline (control) or 100 μg Nle⁴DPhe⁷-α-MSH (NDP-α-MSH) (kindly provided by Dr. Renato Longhi, CNR, Milano, Italy) dissolved in 0.5 ml saline, every 12 h, starting 1 h before transplantation. NDP-α-MSH, a synthetic analog of α-MSH, was used because of its greater stability relative to the natural peptide with which it shares biological effects. The dose of NDP-α-MSH and the route of administration were selected on the basis of previous studies in animal models of inflammation. Lipton J M, Ceriani G, Macaluso A, McCoy D, Cames K, Biltz J, Catania A, “Anti-inflammatory effects of the neuropeptide alpha-MSH in acute, chronic, and systemic inflammation,” Ann. N. Y Acad. Sci., 741:137-148 (1994).

Experiments were performed to determine whether NDP-α-MSH treatment prolongs allograft survival. For this purpose, NDP-α-MSH or saline was administered from the time of transplantation until spontaneous rejection. In such experiments (n=7), rats were sacrificed when there was loss of palpable contractile activity of the graft. Studies were performed focusing on the influence of NDP-α-MSH on graft histopathology and transcripts involved in inflammation and rejection. For these studies, rats (n=6) were sacrificed 1 or 4 days after transplantation. NDP-α-MSH or saline was administered from day 0 until the sacrifice.

The results of the present research on heterotopic heart transplantation in rats shows that allograft histopathology is greatly improved and survival is significantly prolonged by treatment with the anti-inflammatory peptide α-MSH. Data from this study and from other laboratories and other laboratories indicate that hearts transplanted from Brown Norway into Lewis rats are invariably rejected within 6-7 days. Orsenigo R, Gatti S, Latham L, Trezza P, Marelli O, “FK506 and SMS 201-995: effect on heterotopic heart transplantation in rats,” Transplant Proc., 33:554-555 (2001). In this highly mismatched transplantation, graft survival was extended up to 10-11 days with α-MSH treatment, a remarkable increase in duration in non-immunosuppressed transplantation. Wei R Q. Schwartz C F. Lin H. Chen G H. Bolling S F, “Anti-TNF antibody modulates cytokine and MHC expression in cardiac allografts,” J. Surg. Res., 81:123-128 (1999).

Early tissue injury and graft rejection are clearly linked. Fairchild R L, Kobayashi H, Miura M, “Chemokines and the recruitment of inflammatory infiltrates into allografts,” Graft, 3:s24-s31 (2000). In addition to surgical trauma, allografts undergo a period of cold ischemia followed by reperfusion. During the ischemic period, there is an increase in expression of adhesion molecules that cause leukocyte migration and myocyte damage after reperfusion Id.

Reperfusion injury causes marked intragraft production of pro-inflammatory cytokines such as TNF-α and IL-1 within a few hours after transplantation. These cytokines set off an inflammatory cascade with intragraft production of chemokines, such as MCP-1, which exerts further chemoattraction for neutrophils and macrophages. These antigen-independent events induce inflammatory foci that initiate the second inflammatory phase, mainly induced by late chemokines, such as RANTES and interferon y inducible protein (IP-10). Id. These chemokines induce recruitment of potentially destructive cells, including circulating T cells and natural killer cells. Indeed, upon graft infiltration, the primed T cells are activated and mediate destruction of the allograft tissue and acute rejection.

Therefore, modulation of early post-transplant inflammation, targeted at neutrophil recruitment and activation, may be of considerable benefit to downstream events, including infiltration of alloantigen-primed T cells to the allograft tissue. The data presented here demonstrate that early reduction of tissue damage by an α-MSH peptide treatment actually delays allograft rejection in the absence of immunosuppressive therapy. Histopathological and gene expression patterns of allografts from α-MSH treated rats, examined 24 hours after transplantation, revealed substantial benefit over untreated animals. Wei R Q. Schwartz C F. Lin H. Chen G H. Bolling S F, “Anti-TNF antibody modulates cytokine and MHC expression in cardiac allografts,” J. Surg. Res., 81:123-128 (1999). Consistent with previous observations that α-MSH reduces reperfusion injury, there was a significant reduction in signs of endothelial activation. Margination and infiltration of inflammatory cells as well as endothelial swelling were markedly reduced in α-MSH-treated animals. This is a novel observation; all previous experiments on the α-MSH peptides' influences on ischemia and reperfusion injury have been performed in models of warm ischemia, whereas transplantation related ischemia is cold ischemia. Consensually, gene expression of NOS II, TNF-α and IL-1β and adhesion molecules ICAM-1 and VCAM-1 was significantly inhibited.

Of particular interest, there was marked inhibition of ET-1 gene expression. ET-1 is the most potent endogenous vasoconstrictor yet identified and contributes to reperfusion injury, transplant rejection, and several cardiovascular diseases. Geny B, Piquard F, Lonsdorfer J, Haberey P, “Endothelin and heart transplantation,” Cardiovasc. Res., 39:556-562 (1998). Pro-inflammatory cytokines strongly stimulate ET-1 synthesis and release. Resink T J, Hahn A W, Scott Burden T, Powell J, Weber E, Buhler F R, “Inducible endothelin mRNA expression and peptide secretion in cultured human vascular smooth muscle cells,” Biochem. Blophys. Res. Commun, 168:1303-1310 (1990). Therefore, increased endothelin expression in transplantation is likely a consequence of increased cytokine production. Harmful consequences of endothelin increase are both local and systemic. Geny B, Piquard F, Lonsdorfer J, Haberey P, “Endothelin and heart transplantation,” Cardiovasc. Res., 39:556-562 (1998). Local production causes smooth muscle cell proliferation and migration and graft vasculopathy; release into the circulation induces hypertension and renal damage. For this reason, effective endothelin antagonists are actively sought. Benigni A, Remuzzi G, “Endothelin antagonists,” Lancet, 353:133-138 (1999).

α-MSH-associated benefits on allografts persist over time. Indeed, even four days after transplantation, graft histopathological appearance was healthier in treated animals. Chemokine inhibition could be the mechanism underlying such prolonged beneficial effect. As stated above, chemokines contribute to acute rejection by recruiting potentially destructive cells into the allograft. Fairchild R L, Kobayashi H, Miura M, “Chemokines and the recruitment of inflammatory infiltrates into allografts,” Graft, 3:s24-s31 (2000). Expression of the chemokines MCP-1 and RANTES was substantially reduced in allografts from α-MSH treated rats. Both MCP-1 and RANTES are under transcriptional control of NF-κB. Ohmori Y, Schreiber R D, Hamilton T A, “Synergy between interferon-gamma and tumor necrosis factor-alpha in transcriptional activation is mediated by cooperation between signal transducer and activator of transcription 1 and nuclear factor kappa B,” J. Biol. Chem., 272:14899-14907 (1997). Therefore, it is reasonable to believe that inhibition of these molecules by α-MSH treatment occurred through its well-established inhibition of this nuclear factor. MCP-1 is expressed early after transplantation and is a chemoattractant for monocytes, activated T cells, NK cells, and eosinophils. DeVries M E, Ran L, Kelvin D J, “On the edge: the physiological and pathophysiological role of chemokines during inflammatory and immunological responses,” Semin. Immunol., 11:95-104 (1999). Its inhibition by α-MSH treatment likely contributed to early reduction in inflammatory cell infiltration. α-MSH-associated inhibition of RANTES is particularly interesting in that this chemokine is expressed in later stages after transplantation and induces chemotaxis of memory T cells to sites of injury. Schall T J, Bacon K, Toy K J, and Goeddel D V, “Selective attraction of monocytes and T lymphocytes of the memory phenotype by cytokine RANTES,” Nature, 347:669-671 (1990).

Chemokine receptor antagonists are presently under investigation to suppress allograft rejection. Power C A, Proudfoot AEI, “The chemokine system: novel broad-spectrum therapeutic agents,” Curr. Opin. Pharmacol., 1:417-424 (2001). However, this approach has encountered several difficulties because of redundancy in the chemokine system; multiple chemokines interact with the same receptor and several chemokine receptors mediate inflammatory cell recruitment. Power C A, Proudfoot AEI, “The chemokine system: novel broad-spectrum therapeutic agents,” Curr. Opin. Pharmacol., 1:417-424 (2001). The anti-chemoattractant effect of α-MSH is unique in that it combines inhibitory influences on chemokine production with direct inhibition of neutrophil chemotaxis. Alpha-MSH inhibited IL-8- and fMLP-induced chemotaxis of human neutrophils in vitro through an increase in cAMP content in these cells. Catania A, Rajora N, Capsoni F, Minonzio F, Star R A, Lipton J M, “The neuropeptide α-MSH has specific receptors on neutrophils and reduces chemotaxis in vitro,” Peptides, 17:675-679 (1996). Direct inhibitory influences on neutrophils could be very beneficial in the early phases of reperfusion injury in which intragraft margination is prominent.

The beneficial effect of α-MSH treatment in transplantation may not depend solely on anti-inflammatory influences. In addition to reduced expression of cytokines, chemokines, and adhesion molecules, the present data show substantial inhibition of PDGF-B and FasL. This novel observation suggests that α-MSH influences may extend beyond reduction of inflammatory mediators and occur through additional pathways.

The broad effects of α-MSH make this molecule advantageous over other more specific inhibitors of individual mediators. α-MSH does not abolish production of any specific pro-inflammatory mediator but significantly improves inflammatory disorders through down regulation of multiple agents. Catania A, Lipton J M, “α-Melanocyte stimulating hormone in the modulation of host reactions,” Endocr. Rev., 14:564-576 (1993); Lipton J M, Catania A, “Anti-inflammatory actions of the neuro-immunomodulator α-MSH,” Immunol. Today, 18:140-145 (1997); Lipton J M, Ceriani G, Macaluso A, McCoy D, Carnes K, Biltz J, Catania A, “Anti-inflammatory effects of the neuropeptide alpha-MSH in acute, chronic, and systemic inflammation,” Ann. N.Y. Acad. Sci., 741:137-148 (1994). This should be regarded as a positive feature because pro-inflammatory mediators, though potentially harmful, are needed for host defense reactions. Further, relative to other experimental agents used to prolong allograft survival in preclinical studies, α-MSH is very safe and inexpensive. Alpha-MSH, is currently being tested in phase I clinical trials and may soon be available for therapeutic use. The evidence of the present preclinical study is therefore significant to both theory and practice in organ transplantation. Acute toxicity studies indicate that it is well tolerated and does not cause any significant side effect when given in effective doses. (Inventors unpublished observations.)

The beneficial effects of α-MSH observed in experimental transplantation could be even more pronounced in clinical transplantation. Antigen-independent events are more prominent in clinical transplantation. In experimental studies, living, healthy animals are used as donors, whereas in clinical transplantation studies, cadavers are the primary source of organs. These organs undergo the dire consequences of brain death, which induces inflammatory reactions in peripheral organs. Pratschke J, Wilhelm M J, Kusaka M, Basker M, Cooper D K, Hancock W W, Tilney N L, “Brain death and its influence on donor organ quality and outcome after transplantation,” Transplantation, 67:343-348 (1999). Further, the increasing need for transplantable organs compel inclusion of marginal donors in which antigen-independent reactions in the recipient are probably more pronounced. Treatment with α-MSH peptides at the time of transplantation might reduce organ dysfunction.

### EXAMPLE II

Heart grafts, removed from rats on day 1, 4, or at the time of rejection, were sectioned coronally. Two sections were snap-frozen in liquid nitrogen and stored at −80° C. for RNA extraction and RT-PCR assays. One section was fixed in 10% buffered formalin and paraffin-embedded for light microscopy examination.

Blood for nitrite determination was collected from the inferior vena cava in heparinized syringes. Plasma was separated by centrifugation, aliquoted, and stored at −80° C.

In the RNA extraction and PCR portion of the experiment, snap frozen sections weighing approximately 150 mg were used for RNA extraction. Total RNA was isolated using the acid guanidine-thiocyanate/phenol extraction method. Chomczynski P, Sacchi N, “Single-step method of RNA isolation by acid guanidinium thiocyanate-phenol-chloroform extraction,” Anal. Biochem., 162:156-159 (1987) This was followed by two rounds of acid phenol/chloroform extraction and alcohol precipitation. RNA was checked for integrity by agarose gel electrophoresis and quantitated by optical density measurement (260 nm).

mRNA retrotranscription was carried out using oligo-dT[18] primers and M-MLV reverse transcriptase (Clontech, Paolo Alto, Calif.). A fraction of diluted (1:5) cDNA was used as template and PCR-amplified with specific primers. Tori M, Kitagawa-Sakakida S, Li Z, Izutani H, Horiguchi K, Ito T, Matsuda H, Shirakura R, “Initial T-cell activation required for transplant vasculopathy in re-transplanted rat cardiac allografts,” Transplantation, 70:737-746 (2000). To avoid false positive results due to genomic DNA contamination, PCR primer pairs were designed to anneal with specific coding sequences spanning at least one intron. A fraction of total RNA, which had not undergone retrotranscription, was used as positive control for genomic DNA contamination. Amplified products were resolved on agarose gels loaded with ethidium bromide, and evaluated through densitometric analysis using ImageMaster VDS 3.0 software (Amersham Pharmacia Biotech, Uppsala, Sweden). The expression of each inducible transcript was normalized to that of constitutive housekeeping gene for glyceraldehyde-3-phosphate dehydrogenase (GAPDH). Three independent PCR amplification experiments were performed for each transcript. The ratio of each mRNA/GAPDH was calculated and the data are expressed as means±standard error of the mean (SEM).

Histopathological evaluation was then performed. Three μm coronal sections of heart grafts were stained with hematoxylin and eosin. Histopathological lesions were scored by two blinded observers using a 5-point scale according to combined assessments of edema (absent, perivascular/mild interstitial, subendocardial, diffuse interstitial, massive); inflammatory cell infiltration (absent, perivascular, subendocardial, diffuse interstitial, massive), and myocytolysis (absent, focal, massive): 0=minimum damage, 1+=mild damage, 2+=moderate damage, 3+=severe damage, and 4+=maximum damage.

Analysis of graft infiltrating monocytes/macrophages was performed through immunohistochemical detection of ED1-positive cells. Sections were stained using the peroxidase-antiperoxidase (PAP) technique, after incubation with mouse anti-rat ED 1 or control antibody (Santa Cruz Biotech, Santa Cruz, Calif., USA). Slides were counterstained with hematoxylin and eosin.

Nitric oxide determinations were performed. Specifically, plasma nitrite concentration was determined at times of sacrifice as a measure of nitric oxide release. Nitrates (NO₃⁻) were converted into nitrites (NO₂⁻) by treatment of serum with nitrate reductase (Boehringer Mannheim Italia SpA, Milan, Italy). After enzymatic reduction, samples were mixed with equal amounts of Griess reagent (sulfanilamide 1%, napthlethylenediamide 0.1% in phosphoric acid 0.25%). Samples were incubated at room temperature for 10 min and absorbency was measured at 540 nm using a microplate automatic reader.

Statistical analysis was performed using SigmaStat statistical software (Jandel Scientific GmbH, Germany). Values are expressed as mean±SEM. Unpaired Student's t test was used to compare differences between values in histological scores. Difference in median graft survival time was evaluated using the Mann-Whitney Rank Sum Test. A probability value<0.05 was considered statistically significant.

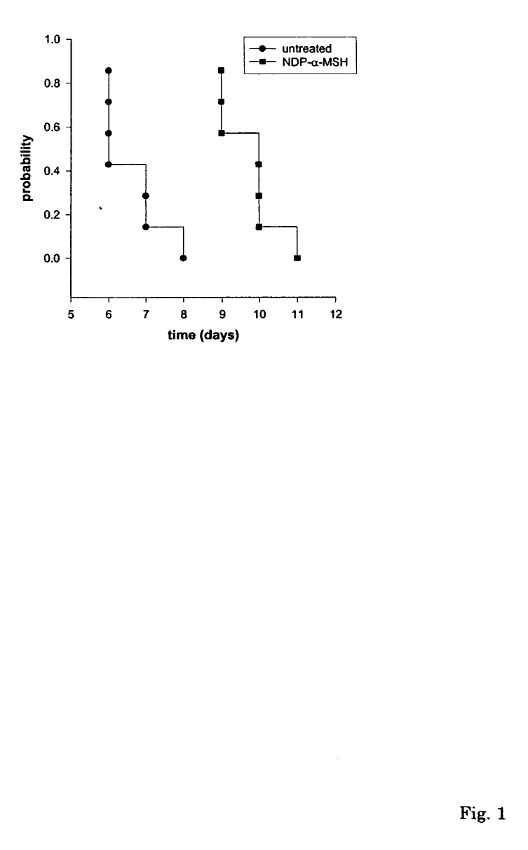

The resulting allograft survival was determined next. Median graft survival in untreated rats was 6 days (range 6-8). Median survival time of the transplanted hearts was significantly prolonged to 10 days (range 9-11) by treatment with NDP-α-MSH (p<0.001) (FIG. 1).

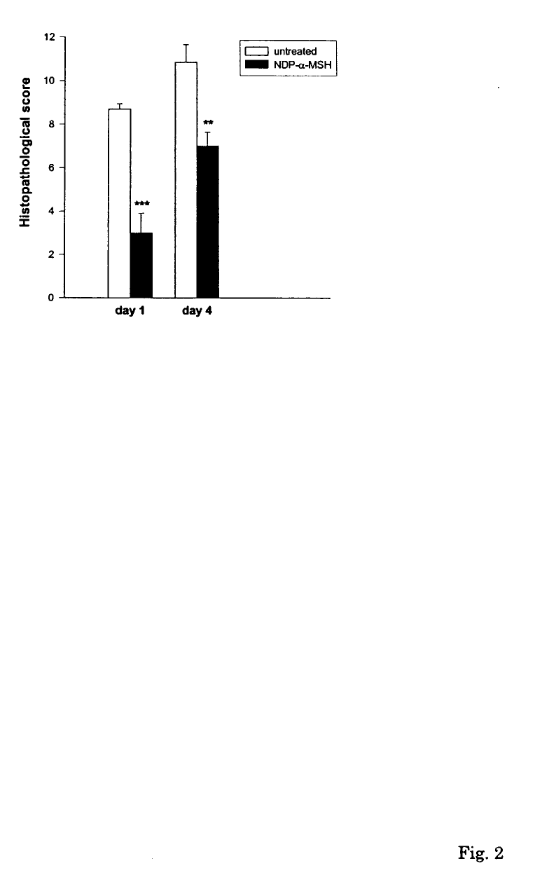

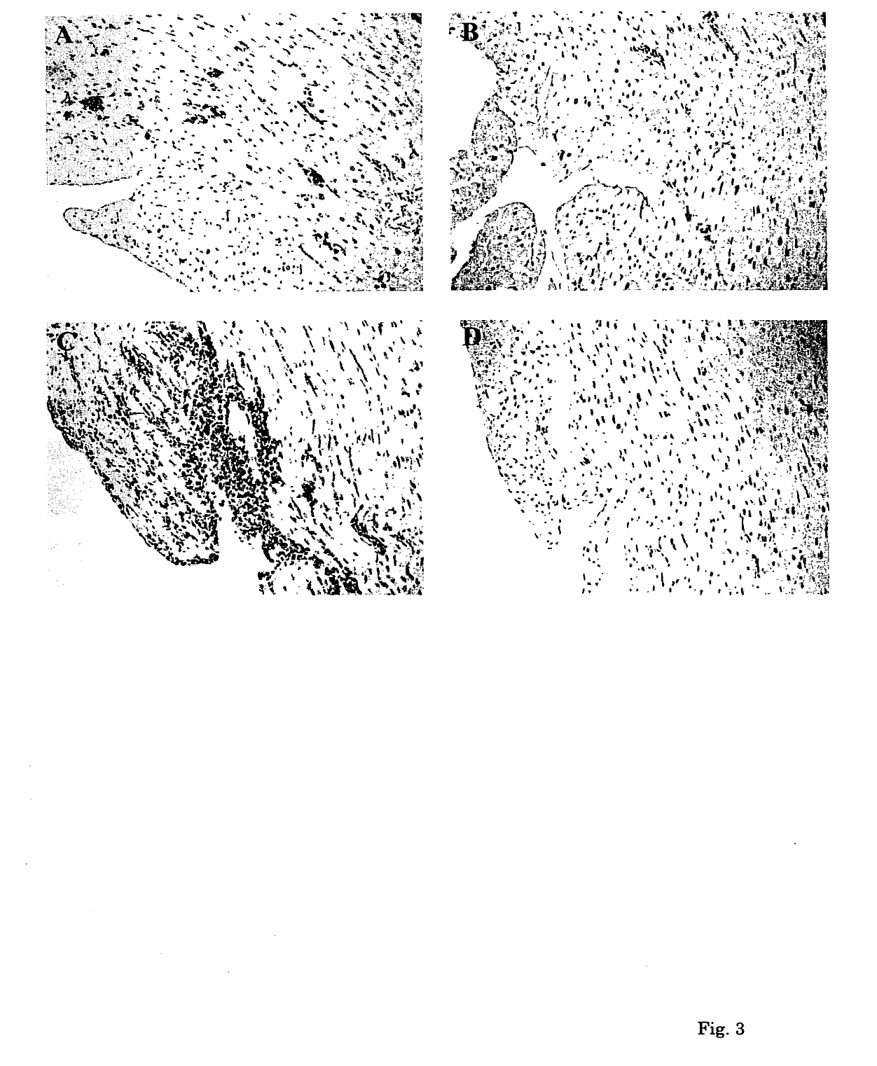

Certain treatment associated histopathological changes were encountered and scored. Histopathological scores of heart grafts harvested on day 1 after transplantation was significantly lower in rats treated with NDP-A-MSH relative to the score in untreated rats (3.0±0.90 vs. 8.7±0.25, mean±SEM; p<0.001) (FIG. 2). Histopathology showed marked interstitial and perivascular edema in untreated rats, whereas intragraft edema was milder and generally restricted to the perivascular spaces in NDP-α-MSH-treated animals. Inflammatory cell infiltration was also much less in hearts of treated animals, with only isolated margination of cells adherent to the endothelia and minimal extravasation. Immunohistochemistry confirmed substantial differences in number and distribution of ED 1-positive cells infiltrating the hearts (FIG. 3).

In hearts harvested 4 days after transplantation, there was still a significant reduction in histopathological score associated with treatment with NDP-α-MSH. Scores were 7.0±0.64 in treated and 10.8±0.80 in untreated animals, respectively, (p<0.01) (FIG. 2). Heart grafts from untreated rats showed diffuse interstitial inflammatory cell infiltration and edema, whereas inflammation and edema were milder and mostly restricted to the subendocardial region in hearts from treated animals. ED1-positive cells were dense and confluent into microabscesses in untreated animals, but fewer and dispersed in hearts of peptide-treated rats (FIG. 3).

There were no significant differences in histology of rejected hearts in treated or untreated animals.

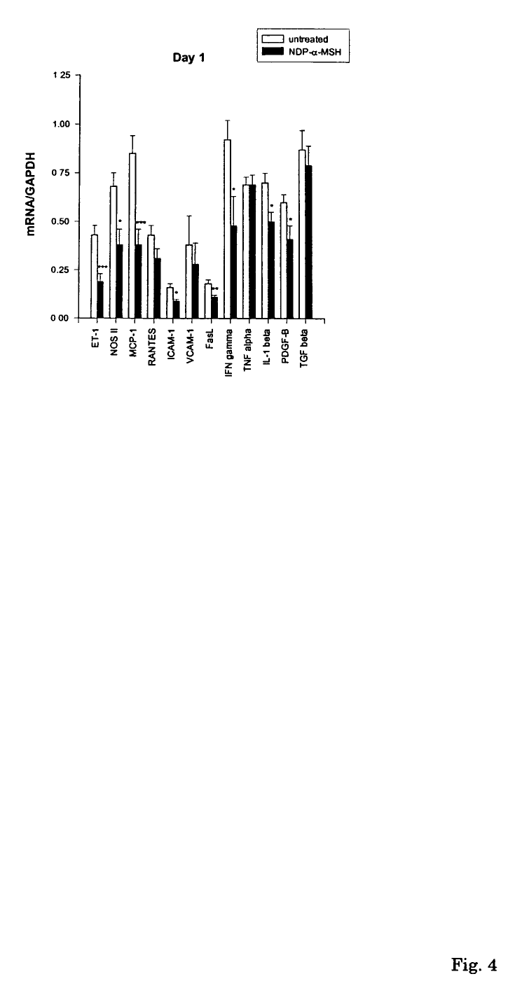

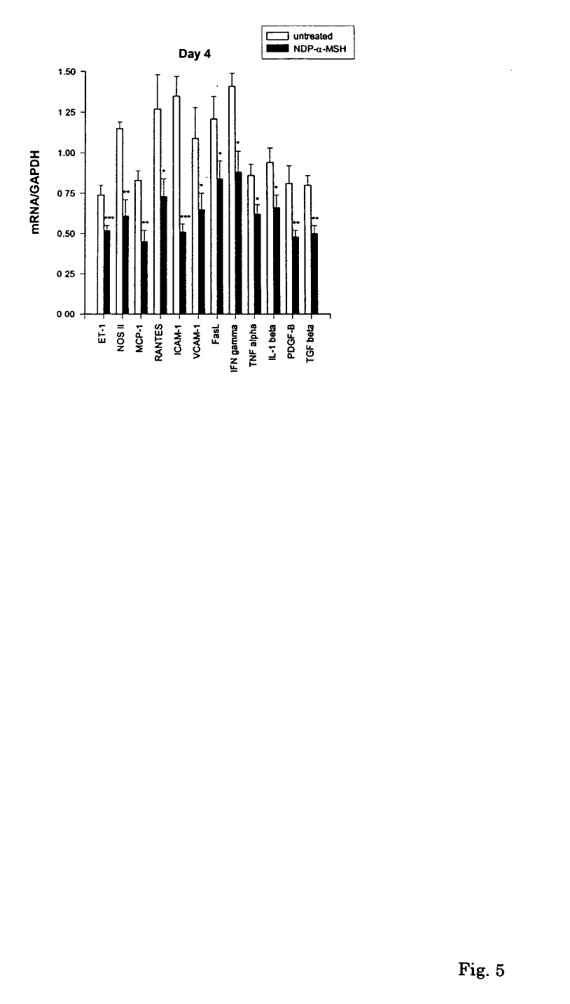

Treatment associated changes in gene expression were determined. Steady state levels of messenger RNA for chemokines, cytokines, adhesion molecules, and growth factors in heart grafts were substantially decreased by α-MSH treatment at both day 1 (FIG. 4) and day 4 (FIG. 5).

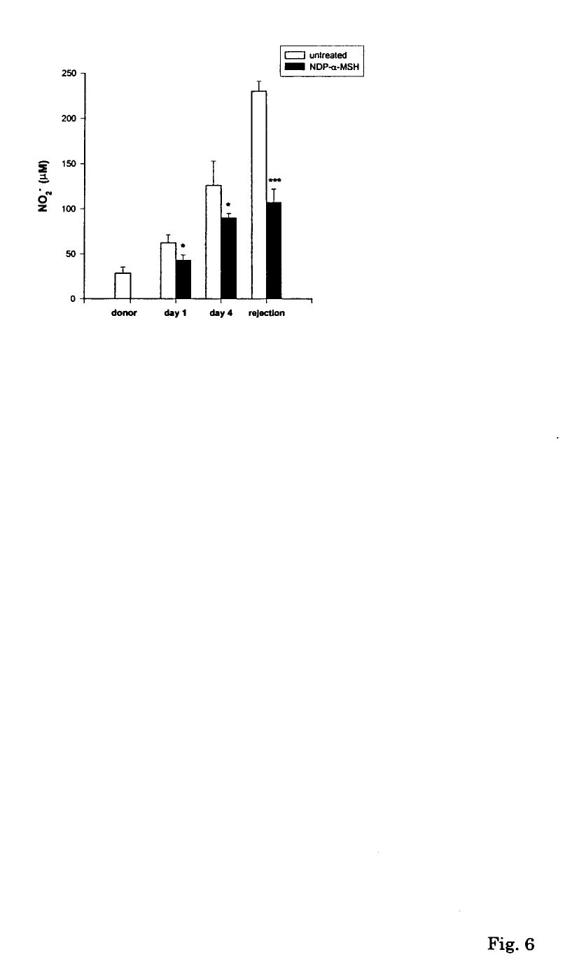

Plasma concentrations of nitrate/nitrite were elevated on day 1 after transplantation relative to concentrations in blood obtained from a donor rat before transplantation (FIG. 6). NO₂⁻ progressively increased, reaching a peak at the time of rejection. In NDP-α-MSH-treated rats, increases were significantly lower at all intervals (FIG. 6). The difference between treated and untreated animals was greatest at the time of rejection.

These experiments and data show the beneficial effects of α-MSH in control of host response to transplantation. This controlled response is apparent in pre-transplant subjects. It follows that additional benefits of the present invention exist with concomitant treatment with immunosuppressive chemotherapy.

The preceding Examples demonstrate benefits to the transplantaion host leading to reduction of transplantation rejection using α-MSH and/or its derivatives. These data are intended only as examples and are not intended to limit the invention to these examples. For example, many α-MSH derivatives, including but not limited to, α-MSH(1-13)(Sequence ID No. 1), NDP-α-MSH(Sequence ID No. 2), α-MSH(11-13)(Sequence ID No. 3) and α-MSH(8-13)(Sequence ID No. 4), are parts of or contain functionally equivilent substitutions within the peptide sequence that markedly increase efficacy and potency. It is understood that modifying the examples above does not depart from the spirit of the invention. It is further understood that each examples can be applied on its own or in combination with each other.

We claim:

1. A method of reducing transplantation rejection, comprising administration of an α-MSH peptide in combination with an immunosuppressive agent within a biologically suitable carrier.

2. The method of claim 1, wherein the α-MSH peptide may be selected from the group consisting of α-MSH(1-13)(Sequence ID No. 1), NDP-α-MSH(Sequence ID No. 2), α-MSH(1-13)(Sequence ID No. 3) and α-MSH(1-13)(Sequence ID No. 4).

3. The method of claim 1 wherein the α-MSH peptide is made up from a sequence of amino acids wherein any of the sequence of amino acids may be of the D-configuration.

4. The method of claim 1, wherein said administration reduces both acute and chronic rejection.

5. The method of claim 1, wherein said administration reduces reperfusion injury.

6. The method of claim 4, wherein the reperfusion injury is cold reperfusion injury.

7. The method of claim 4, wherein the reperfusion injury is warm reperfusion injury.

8. The method of claim 1, wherein said administration prolongs the survival of transplanted tissue.

9. The method of claim 1 wherein the immunosuppressive therapy may be selected from the group consisting of corticosteroids, azathioprine, cyclophosphamide, x-ray irradiation, anti-lymphocyte globulins, anti-thymocyte globulins; cyclosporine; and monoclonal antibodies or combinations thereof.

10. The method of claim 1, werein administration may occur before, during and after transplantion.

11. The method of claim 10, wherein the administration may be selected from the group consisting of oral, anal, parenteral, intravascular, intrarterial, topical, transdermal, vaginal, intratracheobronchial, intraperitoneal, intracerbroventricular and mucosal administration.

12. The method of claim 1, wherein the biologically suitable carrier may be selected from the group consisting of sterile water, propylene glycol, polyethylene glycol, vegetable oils, organic esters, saline, dextrose, mannitol, lactose, lecithin, albumin, sodium glutamate, cysteine hydrochloride, phosphate-buffered saline, Ringer's solution and Ringer's lactate.

13. A method of reducing leukocyte infiltration into transplanted tissue comprising administration of compostion an α-MSH peptide and a biologically suitable carrier.

14. The method of claim 13 wherein the leukocytes are selected from the group containg neutrophins, polymorphnuclear leukocytes, monocytes, activated T-Cells, memory T-Cells, B-Cells, NK Cells, and esinophils.

15. The method of claim 13 wherein the α-MSH peptide may be selected from the group consisting of α-MSH(1-13)(Sequence ID No. 1), NDP-α-MSH(Sequence ID No. 2), α-MSH(1-13)(Sequence ID No. 3) and α-MSH(1-13)(Sequence ID No. 4).

16. The method of claim 13 wherein the α-MSH peptide is made up from a sequence of amino acids wherein any of the sequence of amino acids may be of the D-configuration.

17. The method of claim 13 wherein any of the sequence of amino acids may be in a D-configuration.

18. The method of claim 13, wherein said administration reduces both acute and chronic rejection.

19. The method of claim 13, wherein said administration reduces reperfusion injury.

20. The method of claim 19 wherein the reperfusion injury is cold reperfusion injury.

21. The method of claim 19 wherein the reperfusion injury is warm reperfusion injury.

22. The method of claim 13, wherein said administration prolongs the survival of transplanted tissue.

23. The method of claim 13 werein administration may occur before, during and after transplantion.

24. The method of claim 13, wherein the administration can be selected from the group consisting of oral, anal, parenteral, intravascular, intrarterial, topical, transdermal, vaginal, intratracheobronchial, intraperitoneal, intracerbroventricular and mucosal administration.

25. The method of claim 13 wherein the biologically suitable carrier may be selected from the group consisting of sterile water, propylene glycol, polyethylene glycol, vegetable oils, organic esters, saline, dextrose, mannitol, lactose, lecithin, albumin, sodium glutamate, cysteine hydrochloride, phosphate-buffered saline, Ringer's solution and Ringer's lactate.

26. A method of preventing apoptosis in transplant tissue comprising the administration of a composition comprising an α-MSH peptide and a biologically acceptable carrier.

27. The method of claim 26, wherein said administration reduces both acute and chronic rejection.

28. The method of claim 26 wherein the α-MSH peptide may be selected from the group consisting of α-MSH(1-13)(Sequence ID No. 1), NDP-α-MSH(Sequence ID No. 2), α-MSH(1-13)(Sequence ID No. 3) and α-MSH(1-13)(Sequence ID No. 4).

29. The method of claim 26 wherein the α-MSH peptide is made up from a sequence of amino acids wherein any of the sequence of amino acids may be of the D-configuration.

30. The method of claim 26, wherein said administration reduces reperfusion injury.

31. The method of claim 30, wherein the reperfusion injury is cold reperfusion injury.

32. The method of claim 30 wherein the reperfusion injury is warm reperfusion injury.

33. The method of claim 26, wherein said administration prolongs the survival of transplanted tissue.

34. The method of claim 26 werein administration may occur before, during and after transplantion.

35. The method of claim 34, wherein the administration can be selected from the group consisting of oral, anal, parenteral, intravascular, intrarterial, topical, transdermal, vaginal, intratracheobronchial, intraperitoneal, intracerbroventricular and mucosal administration.

36. The method of claim 35, wherein the biologically suitable carrier may be selected from the group consisting of sterile water, propylene glycol, polyethylene glycol, vegetable oils, organic esters, saline, dextrose, mannitol, lactose, lecithin, albumin, sodium glutamate, cysteine hydrochloride, phosphate-buffered saline, Ringer's solution and Ringer's lactate.

37. A method of reducing reperfusion injury and increasing survival time of transplanted tissue survival time of transplanted tissue comprising the administration of a composition comprising an α-MSH peptide and a biologically acceptable carrier.

38. The method of claim 37 wherein the α-MSH peptide may be selected from the group consisting of α-MSH(1-13)(Sequence ID No. 1), NDP-α-MSH(Sequence ID No.2), α-MSH(1-13)(Sequence ID No.3) and α-MSH(1-13)(Sequence ID No.4).

39. The method of claim 37 wherein the α-MSH peptide is made up from a sequence of amino acids wherein any of the sequence of amino acids may be of the D-configuration.

40. The method of claim 37, wherein said administration reduces both acute and chronic rejection.

41. The method of claim 37 wherein the reperfusion injury is cold reperfusion injury.

42. The method of claim 37 wherein the reperfusion injury is warm reperfusion injury.

43. The method of claim 37 werein administration may occur before, during and after transplantion.

44. The method of claim 43, wherein the administration can be selected from the group consisting of oral, anal, parenteral, intravascular, intrarterial, topical, transdermal, vaginal, intratracheobronchial and mucosal administration.

45. The method of claim 37 wherein the biologically suitable carrier may be selected from the group consisting of sterile water, propylene glycol, polyethylene glycol, vegetable oils, organic esters, saline, dextrose, mannitol, lactose, lecithin, albumin, sodium glutamate, cysteine hydrochloride, phosphate-buffered saline, Ringer's solution and Ringer's lactate.


SEQUENCE LISTING (4 sequences)

SEQ ID NO: 1 - protein, 13 aa, Artificial sequence
  Designed polypeptide with anti-inflammatory, anti-bacterial, anti-fungal and antipyretic properties.
SYSMEHFRWGKPV

SEQ ID NO: 2 - protein, 16 aa, Artificial sequence
  Designed polypeptide with anti-inflammatory, anti-bacterial, anti-fungal and antipyretic properties.
NDPSYSMEHFRWGKPV

SEQ ID NO: 3 - protein, 3 aa, Artificial sequence
  Designed polypeptide with anti-inflammatory, anti-bacterial, anti-fungal and antipyretic properties.
KPV

SEQ ID NO: 4 - protein, 6 aa, Artificial sequence
  Designed polypeptide with anti-inflammatory, anti-bacterial, anti-fungal and antipyretic properties.
RWGKPV